(19) United States
(12) Patent Application Publication
(10) Pub. No.: US 2024/0317843 A1
(43) Pub. Date: Sep. 26, 2024

(54) Methods and Compositions of Thermostabilized Single Domain Antibodies

(71) Applicant: The Government of the United States of America, as represented by the Secretary of the Navy, Arlington, VA (US)

(72) Inventors: Scott N. Dean, Bowie, MD (US); Patricia M. Legler, Derwood, MD (US); Jinny L. Liu, Ellicott City, MD (US)

(21) Appl. No.: 18/609,606

(22) Filed: Mar. 19, 2024

Related U.S. Application Data
(60) Provisional application No. 63/491,169, filed on Mar. 20, 2023.

(51) Int. Cl. C07K 16/12

(52) U.S. Cl. CPC C07K 16/1271; C07K 2317/569; C07K 2317/94; C07K 2319/21

(57) ABSTRACT

Described herein are single domain antibodies (sdAbs) with improved thermostability and methods for the design thereof using a machine learning model (LIME). LIME predicted thermostabilizing mutations were tested in four sets of sequence-related single domain antibodies (sdAb) having binding activity against Staphylococcal Enterotoxin B, 12 sdAb in total. The mutations were restricted to the constant regions. Two sets of interacting residues were consistently predicted. The first set was in the core of three variants, A7D, with W37R or W37H; these were predicted to form a salt bridge. The second predicted set of, interacting residues was on the surface and created a network of hydrogen bonds involving P22T with S8T accompanied by Q6S. Six mutations were common to all four sets: Q6S, A7D, S8T, (R/Q/E)14R, S18R, and P22T.

### CROSS-REFERENCE TO RELATED APPLICATIONS

This application claims the benefit of Provisional U.S. Patent Application No. 63/491,169 filed on Mar. 20, 2023, which is incorporated herein by reference in its entirety.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; nrltechtran@us.navy.mil, referencing NC 211278.

### INCORPORATION BY REFERENCE

This application incorporates by reference the Sequence Listing XML file submitted herewith via the patent office electronic filing system having the file name “211278US2-sequences.xml” and created on Mar. 11, 2024 with a file size of 15,813 bytes.

### BACKGROUND

Single domain antibodies (sdAb) and other antibody products display their merit in a variety of areas such as therapeutics and diagnostics. Antibody engineering has dramatically evolved. Current antibody drugs, for example, have increasingly fewer adverse effects, such that therapeutic antibodies represent a significant fraction of the new drugs developed in recent years.

Engineering thermostability into antibodies or other protein products can extend the product's shelf life and reduce the replenishment costs for stockpiling. In the past, directed evolution has been applied to enhance binding and solubility for some of these heterologous expressed proteins. To date, antibody discovery has remained to be affixed with expensive technologies with minimal yield.

A need exists for techniques for rapidly and inexpensively producing antibodies with improved stability, as well as such antibodies themselves.

### BRIEF SUMMARY

Described herein are single domain antibodies (sdAbs) with improved thermostability and methods for the design thereof using a machine learning model (LIME). LIME predicted thermostabilizing mutations were tested in four sets of sequence-related single domain antibodies (sdAb), 12 sdAb in total. The mutations were restricted to the constant regions. Two sets of interacting residues were consistently predicted. The first set was in the core of three variants, A7D, with W37R or W37H; these were predicted to form a salt bridge. The second predicted set of, interacting residues was on the surface and created a network of hydrogen bonds involving P22T with S8T accompanied by Q6S. Six mutations were common to all four sets: Q6S, A7D, S8T, (R/Q/E)14R, S18R, and P22T. Notably, both sets of mutations were near the disulfide bond in the core of the v-set domain. In variant 1.2, a 12.7 degree increase in the Tₘ was obtained after five mutations. However, similar mutations in variant 18.2 led to a small decrease in Tₘ suggesting that the variable regions of the sdAb may subtly affect the chi angles of the side chains. AlphaFold multimer models show the predicted epitopes on the antibody target.

In a first embodiment, a single domain antibody comprises a sequence of amino acid residues selected from the group consisting of SEQ ID NOS: 2, 3, 5, 6, 8, 9, 11, 12, 13, 14, and 15.

Further embodiment include single domain antibodies according to the first embodiment, wherein the sequence lacks some or all of the DDDDK tag, and/or has an additional terminal hexahistidine tag.

Another embodiment is a single domain antibody comprising the mutations Q6S, A7D, S8T, (R/Q/E)14R, and P22T. Optionally, the antibody further comprises a tag to improve solubility.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B relate to structural models of series 1.1 and 1.2 sdAbs. FIG. 1A shows ML predicted changes at each round. On the surface S8T and P22T are predicted to be within hydrogen bonding distance (red dashed lines). FIG. 1B shows the melting temperatures of 1.1DDDDK and 1.2DDDDK. The mutations significantly increased the Tm by 12.7° C.

FIGS. 2A and 2B depict Alphafold2 predicted epitopes for LIME sdAB with SEB. FIG. 2A shows the crystal structure of TCRSEB-MHC complex (PDB 4C56). FIG. 2B depicts LIME sdAB with SEB models 1.1 and 1.2.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of +10% of that stated.

### Overview

As described herein, machine learning (ML) was used to predict potentially thermostabilizing mutations in single domain antibodies. Namely, the ML tool Local Interpretable Model-Agnostic Explanations (LIME), which finds a representative model through locally weighted approximations of the original model, was used. This technique allows a low-sample size method for generating new antibody sequences from automatically directing mutations at sites that maintain a local fidelity from its original characteristic and preserve the context of the mutations. Once given an initial input sequence, the model then individually predicted thermostabilizing mutations through a XGBoost regressor.

A series of sdAb variants were generated via ML based on four initial input sequences, denoted: 0.1, 1.1, 17.1, and 18.1. Each of these four initial sequences represent previously published sdAbs having binding activity against Staphylococcal Enterotoxin B (SEB). These four sequences were described in two references. The first reference, K. B. Turner et al., “Next-Generation Sequencing of a Single Domain Antibody Repertoire Reveals Quality of Phage Display Selected Candidates,” PLOS One 11, e0149393 (2016) describes the sdAb sequences where 0.1 as used here is termed Ca in the reference and likewise 1.1 is termed Cd there. The second reference, K. B. Turner et al., “Isolation and epitope mapping of staphylococcal enterotoxin B single-domain antibodies,” Sensors (Basel) 14, 10846-10863 (2014), describes the other two original sequences where 17.1 used here the same as is S222-A2 in the reference and likewise 18.1 here is B9-B11 there.

Starting with representative sequence 1.1 as an input to LIME, the model was asked to produce a variant with improved thermostability. This resulted in the generation of variant 1.2 as seen below in Table 1. In practice, these were each produced with a hexahistidine tail at the C-terminal end for purification.

TABLE 1
Single domain antibody sequences

| Variant | Sequence |
|---|---|
| 0.1 | MEVQLQASGGGLVQAGDSLRLPCAASLRTFGSYALGWFRQAPGKEREFVAAISW |
| | SGGDTYADSVKGRFTISRDNAKSTVYLQMNSLEPEDTAVYSCAAVRARYYISKHA |
| | TDYGFWGQGTQVTVSAAAALE [SEQ ID No: 1] |
| 0.2 | MEVQLQDTGGGLVRAGDRLRLTCAASLRTFGSYALGWFRQAPGKEREFVAAIS |
| | WSGGDTYADSVKGRFTISRDNAKSTVYLQMNSLEPEDTAVYSCAAVRARYYISKH |
| | ATDYWFWGQGTQVTVSAAAALE [SEQ ID No: 2] |
| 0.3 | MEVQLSDTGGGLVRAGFRLRLTCAASLRTFGSYALGRFRQAPGKEREFVAAISWS |
| | GGTTYADSVKGRFTISRDNAKSTVYLQMNSLEPEDTAVYSCAAVRARYYVSKHAD |
| | DYWFWGQGTQVTVSAAAALE [SEQ ID No: 3] |
| 1.1 | MEVQLQASGGGLVQAGGSLRLPCAASGRTFGSYAMGWFRQAPGKEREFVAAIS |
| | WSGGDTYADSVKGRFTISRDNAKNTVYLQMNSLEPEDTAVYWCAAVRARYYISK |
| | VAEDYGYWGQGTQVTVSSAAALE [SEQ ID No: 4] |
| 1.2 | MEVQLSDTGGGLVRAGGSLRLTCAASGRTFGSYAMGWFRQAPGKEREFVAAIS |
| | WSGGDTYADSVKGRFTISRDNAKNTVYLQMNSLEPEDTAVYWCAAVRARYYISK |
| | VAEDYGYWGQGTQVTVSSAAALE [SEQ ID No: 5] |
| 1.3 | MEVQLSDTGGGLVRAGGRLRLTCAASGRTFGSYAMGTFRQAPGKEREFVAAISW |
| | SGGVTYADSVKGRFTISRDNAKNTVYLQMNSLEPEDTAVYWCAAVRARAYVSKV |
| | AEDYWYWGQGTQVTVSSAAALE [SEQ ID No: 6] |
| 17.1 | MDVQLQASGGGLVEPGGSLRLSCAASGSAVSIGFMGWHRQAPGKQRERVAQISS |
| | TGIPNYADTVKGRFTISRDNTKNTMYLQMNSLNADDTAVYFCNARLYDGTSAW |
| | GQGTQVTVSSAAALE [SEQ ID No: 7] |
| 17.2 | MDVQLSDTGGGLVRPGGSLRLTCAASGSAVSIGFMGWHRQAPGKQRERVAQISS |
| | TGIPNYADTVKGRFTISRDNTKNTMYLQMNSLNADDTAVYFCNARLYDGTSAW |
| | GQGTQVTVSSAAALE [SEQ ID No: 8] |
| 17.3 | MDVQLSDTGGGLVRAGGRLRLTCAASGSAVSIGFMGHHRQAPGKQRERVAQIS |
| | SSGIPNYADTVKGRFTISRDNTKNTMYLQMNSLNADDTAMYWCNARLYDGTSA |
| | WGQGTQVTVSSAAALE [SEQ ID No: 9] |
| 18.1 | MDVQLQASGGGLVQVGGSLRLSCAASGSTFRIGYMGWYRQAPGKPRELVARISS |
| | GGTTDYLDFVKDRFTISRDNAKNTVYLQMSSLKPEDTAVYYCNVVNYRANEYWG |
| | QGTQVTVSSAAALE [SEQ ID No: 10] |
| 18.2 | MDVQLSDTGGGLVRAGGSLRLTCAASGSTFRIGYMGWYRQAPGKPRELVARISS |
| | GGTTDYLDFVKDRFTISRDNAKNTVYLQMSSLKPEDTAVYYCNVVNYRANEYWG |
| | QGTQVTVSSAAALE [SEQ ID No: 11] |
| 18.3 | MDVQLSDTGGGLVRAGGRLRLTCAASGSTARIGYMGHYRQAPGKPRELVARISS |
| | GGTTDYLDFVKDRVTISRDNAKNTVYLQMASLKPEDTAVYYCNVVNYRANVYW |
| | GQGTQVTVSSAAALE [SEQ ID No: 13] |
| 1.2^{DDDDK} | MEVQLSDTGGGLVRAGGSLRLTCAASGRTFGSYAMGWFRQAPGKEREFVAAIS |
| | WSGGDTYADSVKGRFTISRDNAKNTVYLQMNSLEPEDTAVYWCAAVRARYYISK |
| | VAEDYGYWGQGTQVTVSSAAADDDDKLE [SEQ ID No: 13] |
| 17.2^{DDDDK} | MAMDVQLSDTGGGLVRPGGSLRLTCAASGSAVSIGFMGWHRQAPGKQRERVA |
| | QISSTGIPNYADTVKGRFTISRDNTKNTMYLQMNSLNADDTAVYFCNARLYDGT |
| | SAWGQGTQVTVSSAAADDDDKLE [SEQ ID No: 14] |
| 18.2^{DDDDK} | MAMDVQLSDTGGGLVRAGGSLRLTCAASGSTFRIGYMGWYRQAPGKPRELVAR |
| | ISSGGTTDYLDFVKDRFTISRDNAKNTVYLQMSSLKPEDTAVYYCNVVNYRANEY |
| | WGQGTQVTVSSAAADDDDKLE [SEQ ID No: 15] |

Some of the sequences in the table have a DDDDK appended for improved protein solubility, as discussed below. This feature, when present, was not introduced by the ML model. However, in candidates for improved temperature stability, the model did introduce mutations at S6, D7, T8, R14, and T22. The odds of these five mutations occurring at random is approximately 1 in 3,200,000 combinations. An additional round identified a sixth mutation, S18R.

Solubility was found to be a primary main limitation for experimental testing of the sdAb sequences obtained from the LIME-based ML model, as the model used has no information available to predict solubility. In a competing methodology of sequence generation, directed evolution (DE) methods using libraries generated from random mutagenesis, a large fraction of the total proteins are inactive, where beneficial mutations only occur at a low frequency of 10⁻³. In order to obtain soluble ML-designed sdAbs, a range of properties were investigated with a finding that the pI of the insoluble proteins was near 9, while the pI of the soluble proteins was <8.6. By adding an enterokinase cleavage site (DDDDK) to the C-terminus, the pI was lowered and the solubility of the ML-predicted sdAb sequences during periplasmic expression in E. coli was found to be enhanced. Thus, several of the ML-generated sequences were modified post generation with the addition of DDDDK.

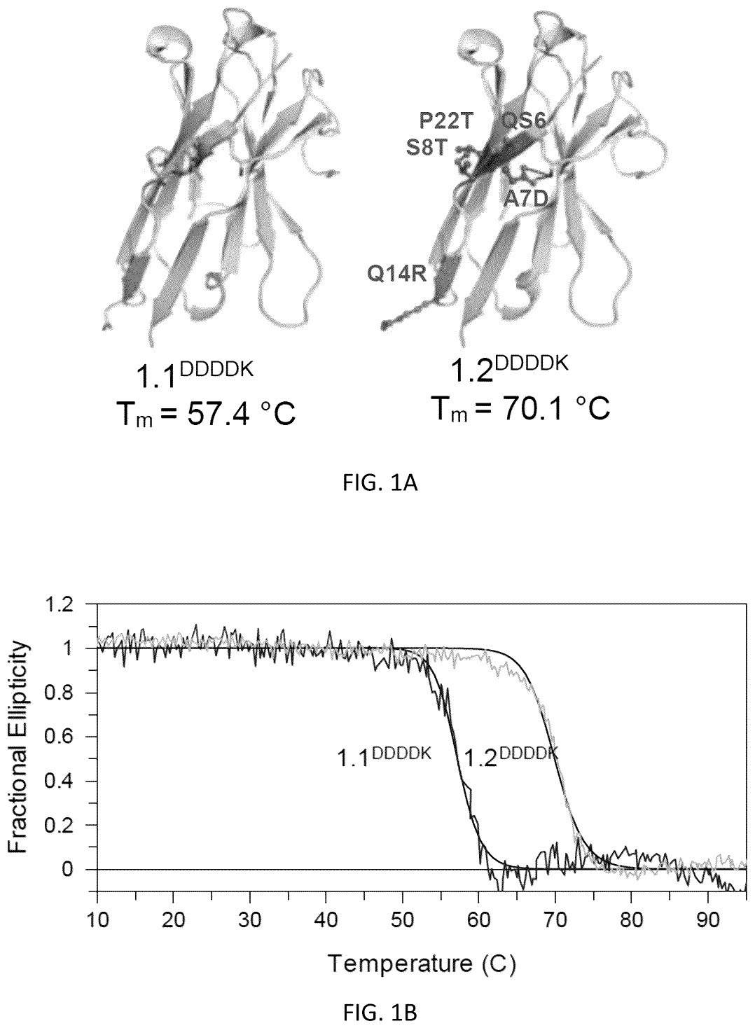

The expressed proteins were measured for thermal denaturation, monitoring 2° C. per minute from 10° C. to 95° C. using a Jasco 810 Circular Dichroism (CD) spectropolarimeter fitted with a Peltier temperature control unit. The melting temperature (Tₘ) was determined from a four-parameter fit of the ellipticity at 205 or 207 nm versus temperature. For Tₘ measurements, the buffer was exchanged to 1×PBS pH 7.4 using PD-10 columns. A protein concentration of 7-14 μM and a 1 mm cuvette were used for CD analysis. Dye melt experiments were used as another method to measure the Tₘ. Here SYPRO Orange Protein Gel Stain (Sigma) was diluted 1000-fold in a final volume of 20 μl PBS with a final protein concentration was 500 μg/ml. Fluorescence was measured using a StepOne Real-Time PCR system (Applied Biosystems) as the sdAbs were heated from 25° C. to 99° C. at a rate of 1% using the ROX channel. It was found that three of the mutation series produced sufficiently soluble protein, namely 1.1^{DDDDK} through 1.2^{DDDDK}, 17.1^{DDDDK} through 17.2^{DDDDK}, and 18.1^{DDDDK} through 18.2^{DDDDK}. The 1.2^{DDDDK} and 17.2^{DDDDK variants} each showed increases in Tₘ 12.7 and 2.9° C., respectively. No significant change was observed in the Tₘ of the 18.1 to 18.2 variants. The mutations and CD denaturation curves for 1.1^{DDDDK} and 1.2^{DDDDK} are shown in FIG. 1. Enhanced solubility can improve homogeneity and recovery of correctly folded protein.

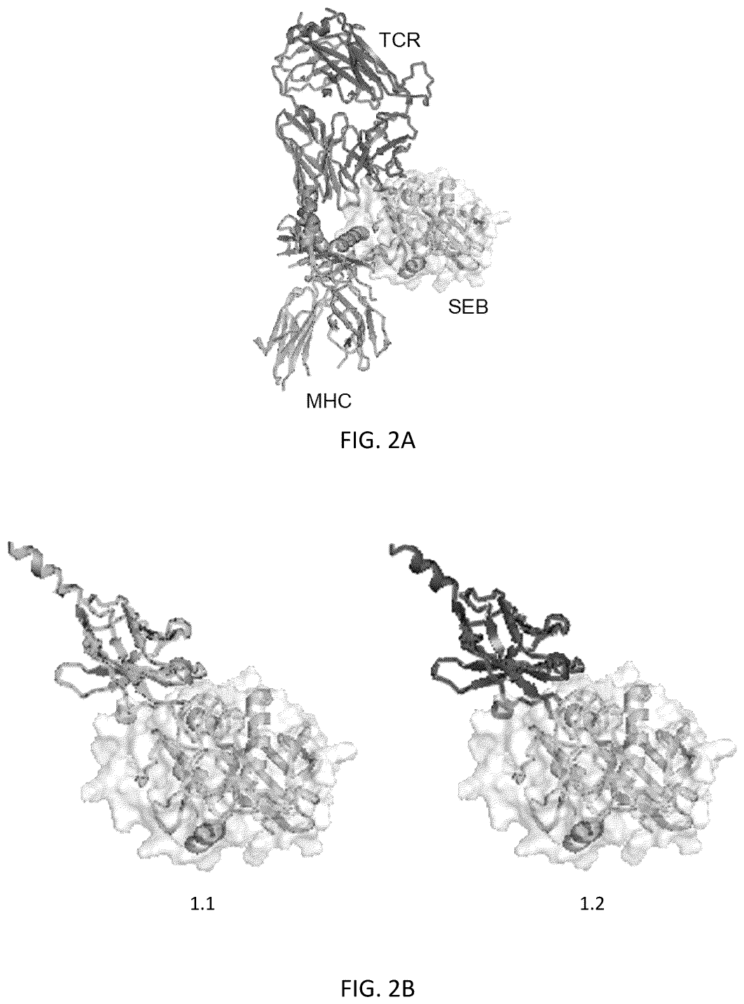

Next, to determine whether the ability to bind to SEB was retained following mutation, surface plasmon resonance (SPR) and the AlphaFold2 model (which predicts the three-dimensional structure of a protein based on its amino acid sequence) were employed. SPR was performed using a ProteOn XPR36 Protein Interaction Array System (Bio-Rad) on a GLC sensor chip. Immobilization of SEB at was first applied to at 20 μg/mL on all lanes of the chip. The binding kinetics of each sdAb was determined by rotating the chip and flowing various concentrations (300, 100, 33, 11, 3.7, 0 nM) over the chip at 100 μL/minute in the orthogonal direction for 90 s over the SEB-coated chip and then monitoring dissociation for 600 s. AlphaFold2 was also used to predict the epitope recognized by the sdAb. Here, models of the antibody-antigen complexes were generated from sequence with AlphaFold2. The predicted sdAb-SEB interfaces overlap predominantly with the binding surface of the TCR (PDB 4C56), but notably not to any unexpected regions of the SEB superantigen (FIGS. 2A and 2B).

### FURTHER EMBODIMENTS

It is suspected that, besides the antibodies disclosed in the examples, sdAbs having one or more of the mutations Q6S, A7D, S8T, (R/Q/E)14R, S18R, and P22T would also exhibit improved thermal stability. To maximize thermal stability, most or all of these mutations should be used. The S18R mutation may be less important, thus, in one embodiment, an sdAb has the mutations Q6S, A7D, S8T, (R/Q/E)14R, and P22T.

While the tested sequences include hexahistidine tags, also contemplated are variants where the histidine tag is of a different length or is absent or is substituted with a different tag useful for protein purification. Similarly contemplated are variants lacking the DDDDK tag for solubility, or having a different tag for improved solubility.

### Advantages

The reported increases in the sdAb's Tm values were obtained using a set of novel mutations while retaining SEB-binding. The mutations increased the Tₘ of two different sdAb (1.2 and 17.2).

The Tₘ of the sdAb is significantly above body temperature (37° C.); thus, these sdAb could be used for coating medical devices such as catheters or for shelf-stable refrigeration free diagnostics such as lateral flow tests.

The sdAb design strategy described here has a range of advantages relative to prior art. This methodology uses a ML model and subsequent modification (adding the DDDDK solubility tag) to arrive at new sdAb candidates to characterize, avoiding the need for the labor-intensive and costly repetitive rounds of sequential mutation experiments. The solubility tag also eliminated misfolding states from inclusion bodies and enhanced recovery of soluble folded protein by >10-fold for the 1.2^{DDDDK} variant when compared with the 1.1^{DDDDK} variant. Next, the method restricted mutations to constant regions of the sdAb, avoiding the variable CDRs, regions that would be affected via the random mutagenesis used in directed evolution experiments. Finally, the method for obtaining the described sequences is nonspecific to SEB-binding sdAbs and can be applied to a variety of inputs.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

What is claimed is:

1. A single domain antibody comprising an amino acid reside sequence selected from the group consisting of SEQ ID NOs: 2, 3, 5, 6, 8, 9, 11, 12, 13, 14, and 15.

2. The single domain antibody of claim 1, further comprising a polyhistidine tag.

3. A single domain antibody comprising each of the mutations Q6S, A7D, S8T, (R/Q/E)14R, and S18R.

4. The single domain antibody of claim 3, further comprising a polyhistidine tag.


SEQUENCE LISTING (15 sequences)

SEQ ID NO: 1 - protein, 130 aa, Synthetic construct
MEVQLQASGGGLVQAGDSLRLPCAASLRTFGSYALGWFRQAPGKEREFVAAISWSGGDTYADSVKGRFTISRDNAKSTVYLQMNSLEPEDTAVYSCAAVRARYYISKHATDYGFWGQGTQVTVSAAAALE

SEQ ID NO: 2 - protein, 130 aa, Synthetic construct
MEVQLQDTGGGLVRAGDRLRLTCAASLRTFGSYALGWFRQAPGKEREFVAAISWSGGDTYADSVKGRFTISRDNAKSTVYLQMNSLEPEDTAVYSCAAVRARYYISKHATDYWFWGQGTQVTVSAAAALE

SEQ ID NO: 3 - protein, 130 aa, Synthetic construct
MEVQLSDTGGGLVRAGFRLRLTCAASLRTFGSYALGRFRQAPGKEREFVAAISWSGGTTYADSVKGRFTISRDNAKSTVYLQMNSLEPEDTAVYSCAAVRARYYVSKHADDYWFWGQGTQVTVSAAAALE

SEQ ID NO: 4 - protein, 130 aa, Synthetic construct
MEVQLQASGGGLVQAGGSLRLPCAASGRTFGSYAMGWFRQAPGKEREFVAAISWSGGDTYADSVKGRFTISRDNAKNTVYLQMNSLEPEDTAVYWCAAVRARYYISKVAEDYGYWGQGTQVTVSSAAALE

SEQ ID NO: 5 - protein, 130 aa, Synthetic construct
MEVQLSDTGGGLVRAGGSLRLTCAASGRTFGSYAMGWFRQAPGKEREFVAAISWSGGDTYADSVKGRFTISRDNAKNTVYLQMNSLEPEDTAVYWCAAVRARYYISKVAEDYGYWGQGTQVTVSSAAALE

SEQ ID NO: 6 - protein, 130 aa, Synthetic construct
MEVQLSDTGGGLVRAGGRLRLTCAASGRTFGSYAMGTFRQAPGKEREFVAAISWSGGVTYADSVKGRFTISRDNAKNTVYLQMNSLEPEDTAVYWCAAVRARAYVSKVAEDYWYWGQGTQVTVSSAAALE

SEQ ID NO: 7 - protein, 122 aa, Synthetic construct
MDVQLQASGGGLVEPGGSLRLSCAASGSAVSIGFMGWHRQAPGKQRERVAQISSTGIPNYADTVKGRFTISRDNTKNTMYLQMNSLNADDTAVYFCNARLYDGTSAWGQGTQVTVSSAAALE

SEQ ID NO: 8 - protein, 122 aa, Synthetic construct
MDVQLSDTGGGLVRPGGSLRLTCAASGSAVSIGFMGWHRQAPGKQRERVAQISSTGIPNYADTVKGRFTISRDNTKNTMYLQMNSLNADDTAVYFCNARLYDGTSAWGQGTQVTVSSAAALE

SEQ ID NO: 9 - protein, 122 aa, Synthetic construct
MDVQLSDTGGGLVRAGGRLRLTCAASGSAVSIGFMGHHRQAPGKQRERVAQISSSGIPNYADTVKGRFTISRDNTKNTMYLQMNSLNADDTAMYWCNARLYDGTSAWGQGTQVTVSSAAALE

SEQ ID NO: 10 - protein, 122 aa, Synthetic construct
MDVQLQASGGGLVQVGGSLRLSCAASGSTFRIGYMGWYRQAPGKPRELVARISSGGTTDYLDFVKDRFTISRDNAKNTVYLQMSSLKPEDTAVYYCNVVNYRANEYWGQGTQVTVSSAAALE

SEQ ID NO: 11 - protein, 122 aa, Synthetic construct
MDVQLSDTGGGLVRAGGSLRLTCAASGSTFRIGYMGWYRQAPGKPRELVARISSGGTTDYLDFVKDRFTISRDNAKNTVYLQMSSLKPEDTAVYYCNVVNYRANEYWGQGTQVTVSSAAALE

SEQ ID NO: 12 - protein, 122 aa, Synthetic construct
MDVQLSDTGGGLVRAGGRLRLTCAASGSTARIGYMGHYRQAPGKPRELVARISSGGTTDYLDFVKDRVTISRDNAKNTVYLQMASLKPEDTAVYYCNVVNYRANVYWGQGTQVTVSSAAALE

SEQ ID NO: 13 - protein, 135 aa, Synthetic construct
MEVQLSDTGGGLVRAGGSLRLTCAASGRTFGSYAMGWFRQAPGKEREFVAAISWSGGDTYADSVKGRFTISRDNAKNTVYLQMNSLEPEDTAVYWCAAVRARYYISKVAEDYGYWGQGTQVTVSSAAADDDDKLE

SEQ ID NO: 14 - protein, 129 aa, Synthetic construct
MAMDVQLSDTGGGLVRPGGSLRLTCAASGSAVSIGFMGWHRQAPGKQRERVAQISSTGIPNYADTVKGRFTISRDNTKNTMYLQMNSLNADDTAVYFCNARLYDGTSAWGQGTQVTVSSAAADDDDKLE

SEQ ID NO: 15 - protein, 129 aa, Synthetic construct
MAMDVQLSDTGGGLVRAGGSLRLTCAASGSTFRIGYMGWYRQAPGKPRELVARISSGGTTDYLDFVKDRFTISRDNAKNTVYLQMSSLKPEDTAVYYCNVVNYRANEYWGQGTQVTVSSAAADDDDKLE